(19) United States
(12) Patent Application Publication
(10) Pub. No.: US 2005/0197376 A1
(43) Pub. Date: Sep. 8, 2005

(54) Concomitant drugs

(75) Inventors: Hiroshi Kayakiri, Osaka (JP); Takeshi Kato, Osaka (JP); Hideaki Minoura, Osaka (JP); Jiro Hirosumi, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Fujisawa Pharmaceutical Co. Ltd., Osaka-shi (JP)

(21) Appl. No.: 11/068,747

(22) Filed: Mar. 2, 2005

(30) Foreign Application Priority Data
Mar. 2, 2004 (JP) 057555/2004

(51) Int. Cl. A61K 31/4184; A61K 31/405; A61K 31/343

(52) U.S. Cl. 514/394; 514/419; 514/414; 514/469

(57) ABSTRACT

This invention provides a pharmaceutical agent containing, in combination, a sulfonamide compound and other therapeutic agent, preferably, at least one compound represented by the formula (I): R¹—SO₂—NH—CO-A¹-CH₂—R² [each symbol is as defined in the specification] or a pharmaceutically acceptable salt thereof, and at least one pharmaceutical agent selected from the group consisting of an α-glucosidase inhibitor, an insulin secretagogue, a sulfonylurea and a biguanide, which has a superior therapeutic effect.

### TECHNICAL FIELD OF THE INVENTION

The present invention relates to a pharmaceutical agent comprising a sulfonamide compound and at least one kind of a therapeutic agent other than the compound in combination.

### BACKGROUND OF THE INVENTION

Diabetes is a metabolic disease characterized by hyperglycemia and insulin resistance, which is a chronic disease sometimes causing complications such as obesity, hypertension, hyperlipidemia, cardiovascular disorder, retinopathy and the like. Therefore, it is necessary to select a pharmaceutical agent suitable for the condition of individual diabetic patients. However, single use of each individual pharmaceutical agent often fails to provide a sufficient effect.

In recent years, the pathology of diabetes has been elucidated and, in parallel, pharmaceutical agents having new mechanism of action have appeared. For example, a sulfonamide compound having a basic skeleton of —SO₂—NH—CO— has been found to have a hypoglycemic action and be effective for impaired glucose tolerance, diabetes, diabetic complications, insulin resistance syndrome and the like (WO97/24334, WO98/15530, WO99/00359, WO99/00372, WO99/00373, WO99/51574, WO00/34277, WO00/39097 and WO00/39099).

However, these references do not disclose that a combined use of a sulfonamide compound having a basic skeleton of —SO₂—NH—CO— and other pharmaceutical agent as in the present invention provides a more superior treatment effect than that provided by an individual use thereof.

Each reference is hereby incorporated by the disclosure of the source thereof.

### SUMMARY OF THE INVENTION

The present invention aims at provision of a pharmaceutical agent capable of effectively preventing or treating diseases such as impaired glucose tolerance disorder, diabetes (e.g., type II diabetes), gestational diabetes, diabetic complications (e.g., diabetic gangrene, diabetic arthropathy, diabetic osteopenia, diabetic glomerulosclerosis, diabetic nephropathy, diabetic dermatopathy, diabetic neuropathy, diabetic cataract, diabetic retinopathy and the like), insulin resistance syndrome (e.g., insulin receptor abnormality, Rabson-Mendenhall syndrome, leprechaunism, Kobberling-Dunnigan syndrome, Lawrence-Seip syndrome (adipose tissue atrophy), Gushing syndrome, acromegaly and the like), polycystic ovary syndrome, hyperlipidemia, atherosclerosis, cardiovascular diseases (e.g., stenocardia, cardiac failure and the like), hyperglycemia (e.g., those characterized by abnormal saccharometabolism such as eating disorders), pancreatitis, osteoporosis, hyperuricemia, hypertension, inflammatory bowel diseases, and skin disorders related to an anomaly of differentiation of epidermic cells and the like.

In view of the above-mentioned problems, the present inventors have conducted intensive studies and found that a superior prophylactic or therapeutic effect on various diseases exemplified above can be afforded by combining a sulfonamide compound having a hypoglycemic action as an essential component with other therapeutic drug having different action mechanism, and that the administration amount thereof can be reduced or side effects, such as edema, body weight gain and the like, can be reduced as compared to a single administration of each drug, which resulted in the completion of the present invention.

In summary, the present invention provides the following.
[1] A pharmaceutical agent comprising, in combination, at least one compound represented by the formula (I):
   R¹—SO₂—NH—CO-A₁-CH₂—R¹ (I)
   wherein
R¹ is a group selected from the group consisting of an optionally substituted lower alkyl group, an optionally substituted lower alkenyl group, an optionally substituted aryl group and an optionally substituted heterocyclic group,
R² is an optionally substituted aryl group, or an optionally substituted heterocyclic group, and
A¹ is an optionally substituted divalent group of heterobicycle, or a group represented by —CH═CH-A²- wherein A² is an optionally substituted divalent group of nitrogen-containing 5- or 6-membered heterocycle,
or a pharmaceutically acceptable salt thereof, and at least one pharmaceutical agent selected from the group consisting of an α-glucosidase inhibitor, an insulin secretagogue, a sulfonylurea and a biguanide.
[2] The pharmaceutical agent of the above-mentioned [1], wherein R¹ is a lower alkyl group; a lower alkenyl group optionally substituted by an aryl group; an aryl group optionally substituted by a lower alkyl group or a lower alkenyl group; or a heterocyclic group optionally substituted by a halogen atom, and
R² is an aryl group or a heterocyclic group, which group is substituted by at least one group selected from the group consisting of an optionally substituted lower alkyl group, an optionally substituted lower alkenyl group, an optionally substituted lower alkynyl group, an optionally substituted lower alkoxy group, a lower alkylthio group, an optionally substituted amino group, a morpholino group, an acyl group, a halogen atom, an aryl group, an optionally substituted heterocyclic group, and a nitro group.
[3] The pharmaceutical agent of the above-mentioned [1], wherein R¹ is a lower alkyl group, a lower alkenyl group optionally substituted by a phenyl group, a phenyl group optionally substituted by a lower alkyl group or a lower alkenyl group or a halothienyl group,
R² is a group selected from the group consisting of a phenyl group, a naphthyl group and pyridyl group, which group is substituted by at least one group selected from the group consisting of a lower alkyl group optionally substituted by a halogen atom, a phenyl group, a phenoxy group or a cyclo(lower)alkyloxy group; a lower alkenyl group; a lower alkynyl group optionally substituted by a phenyl group; a lower alkoxy group optionally substituted by a phenyl group, a cyclo(lower)alkyl group or a thienyl group; a lower alkylthio group; a lower alkylamino group optionally substituted by a lower alkoxycarbonyl group; a lower alkanoylamino group; a lower alkoxycarbonyl group; a halogen atom; a phenyl group; a thienyl group optionally substituted by a halogen atom; a furyl group; a morpholino group; and a nitro group, and
A¹ is a divalent group of heterobicycle selected from the group consisting of benzimidazole, indole, imidazopyridine, benzofuran and indazole, which group is substituted by at least one lower alkyl group; or A¹ is a group represented by —CH═CH-A²- wherein A² is a divalent group of imidazole, which group is a substituted by at least one group selected from the group consisting of a lower alkyl group and a halogen atom.
[4] The pharmaceutical agent of the above-mentioned [1], which comprises a compound represented by the formula (I) and an α-glucosidase inhibitor in combination.
[5] The pharmaceutical agent of the above-mentioned [4], wherein the α-glucosidase inhibitor is selected from the group consisting of miglitol, voglibose, miglustat, acarbose, and celgosivir hydrochloride.
[6] The pharmaceutical agent of the above-mentioned [1], which comprises a compound represented by the formula (I) and an insulin secretagogue in combination.
[7] The pharmaceutical agent of the above-mentioned [6], wherein the insulin secretagogue is selected from the group consisting of nategnide, glimepiride, repaglinide, glisentide, mitiglinide, glucagons-like peptide-17-36-amide, glucagons-like peptide-1-amylin and CJC1131.
[8] The pharmaceutical agent of the above-mentioned [1], which comprises a compound represented by the formula (I) and a sulfonylurea in combination.
[9] The pharmaceutical agent of the above-mentioned [8], wherein the sulfonylurea is selected from the group consisting of limepiride and glisentide.
[10] The pharmaceutical agent of the above-mentioned [1], which comprises a compound represented by the formula (I) and a biguanide in combination.
[11] The pharmaceutical agent of the above-mentioned [10], wherein the biguanide is selected from the group consisting of phenformin, metformin and buformin.
[12] A method for preventing and/or treating impaired glucose tolerance disorder, diabetes, gestational diabetes, diabetic complications, insulin resistance syndrome, polycystic ovary syndrome, hyperlipidemia, atherosclerosis, cardiovascular diseases, hyperglycemia, pancreatitis, osteoporosis, hyperuricemia, hypertension, inflammatory bowel diseases, or skin disorders related to an anomaly of differentiation of epidermic cells in a mammal in need thereof, which comprises administering an effective amount of at least one compound represented by the formula (I):
   R₁—SO₂—NH—CO-A₁-CH₂—R² (I)
   wherein
R¹ is a group selected from the group consisting of an optionally substituted lower alkyl group, an optionally substituted lower alkenyl group, an optionally substituted aryl group and an optionally substituted heterocyclic group,
R² is an optionally substituted aryl group or an optionally substituted heterocyclic group, and
A¹ is an optionally substituted divalent group of heterobicycle or a substituent represented by —CH═CH-A²-, wherein A² is an optionally substituted divalent group of nitrogen-containing 5- or 6-membered heterocycle,
   or a pharmaceutically acceptable salt thereof, to said mammal, and administering, to said mammal, an effective amount of at least one pharmaceutical agent selected from the group consisting of an α-glucosidase inhibitor, an insulin secretagogue, a sulfonylurea and a biguanide.
[13] A method of administering, to a mammal, an effective amount of at least one compound represented by the formula (I):
   R¹—SO₂—NH—CO-A₁-CH₂—R² (I)
   wherein
R¹ is a group selected from the group consisting of an optionally substituted lower alkyl group, an optionally substituted lower alkenyl group, an optionally substituted aryl group and an optionally substituted heterocyclic group,
R² is an optionally substituted aryl group or an optionally substituted heterocyclic group, and
A¹ is an optionally substituted divalent group of heterobicycle or a substituent represented by —CH═CH-A²-, wherein A² is an optionally substituted divalent group of nitrogen-containing 5- or 6-membered heterocycle,
   or a pharmaceutically acceptable salt thereof, and an effective amount of at least one pharmaceutical agent selected from the group consisting of an α-glucosidase inhibitor, an insulin secretagogue, a sulfonylurea and a biguanide,
which comprises administering said compound and said pharmaceutical agent simultaneously, separately or in a staggered manner.
[14] Use of at least one compound represented by the formula (I):
   R¹—SO₂—NH—CO-A₁-CH₂—R¹ (I)
   wherein
R¹ is a group selected from the group consisting of an optionally substituted lower alkyl group, an optionally substituted lower alkenyl group, an optionally substituted aryl group and an optionally substituted heterocyclic group,
R² is an optionally substituted aryl group or an optionally substituted heterocyclic group, and
A¹ is an optionally substituted divalent group of heterobicycle or a substituent represented by —CH═CH-A²-, wherein A² is an optionally substituted divalent group of nitrogen-containing 5- or 6-membered heterocycle,
   or a pharmaceutically acceptable salt thereof, and at least one pharmaceutical agent selected from the group consisting of an α-glucosidase inhibitor, an insulin secretagogue, a sulfonylurea and a biguanide for the production of an agent for the prophylaxis or treatment of impaired glucose tolerance disorder, diabetes, gestational diabetes, diabetic complications, insulin resistance syndrome, polycystic ovary syndrome, hyperlipidemia, atherosclerosis, cardiovascular diseases, hyperglycemia, pancreatitis, osteoporosis, hyperuricemia, hypertension, inflammatory bowel diseases or skin disorders related to an anomaly of differentiation of epidermic cells.
[15] A commercial package comprising a pharmaceutical agent comprising, in combination, at least one compound represented by the formula (I):
   R₁—SO₂—NH—CO-A₁-CH₂—R² (I)
   wherein
R¹ is a group selected from the group consisting of an optionally substituted lower alkyl group, an optionally substituted lower alkenyl group, an optionally substituted aryl group and an optionally substituted heterocyclic group,
R² is an optionally substituted aryl group or an optionally substituted heterocyclic group, and
A¹ is an optionally substituted divalent group of heterobicycle or a substituent represented by —CH═CH-A²-, wherein A² is an optionally substituted divalent group of nitrogen-containing 5- or 6-membered heterocycle,
   or a pharmaceutically acceptable salt thereof, and at least one pharmaceutical agent selected from the group consisting of an α-glucosidase inhibitor, an insulin secretagogue, a sulfonylurea and a biguanide, and
a written matter associated with the pharmaceutical agent, the written matter stating that the pharmaceutical agent can or should be used for preventing or treating impaired glucose tolerance disorder, diabetes, gestational diabetes, diabetic complications, insulin resistance syndrome, polycystic ovary syndrome, hyperlipidemia, atherosclerosis, cardiovascular diseases, hyperglycemia, pancreatitis, osteoporosis, hyperuricemia, hypertension, inflammatory bowel diseases or skin disorders related to an anomaly of differentiation of epidermic cells.

The sulfonamide compound to be used in the present invention is not particularly limited as long as the compound has a hypoglycemic action and a basic skeleton of —SO₂—NH—CO—, but a sulfonamide compound described in WO97/24334, WO98/15530, WO99/00359, WO99/00372, WO99/00373, WO99/51574, WO00/34277, WO00/39097 and WO00/39099 or a pharmaceutically acceptable salt thereof is preferable. More particularly, a compound represented by the formula (I):
R₁—SO₂—NH—CO-A₁-CH₂—R² (I)
or a pharmaceutically acceptable salt thereof (hereinafter sometimes to be simply referred to collectively as a sulfonamide compound) is preferable.

In the formula (I), R¹ is an optionally substituted lower alkyl group, an optionally substituted lower alkenyl group, an optionally substituted aryl group or an optionally substituted heterocyclic group.

In the formula (I), R² is an optionally substituted aryl group or an optionally substituted heterocyclic group.

The “lower alkyl group” for R¹ is a linear or branched alkyl group having 1 to 6 carbon atoms. As specific examples, for example, methyl, ethyl, 1-propyl, i-propyl, 1-butyl, i-butyl, tert-butyl, sec-butyl, 1-pentyl, i-pentyl, sec-pentyl, tert-pentyl, methylbutyl, 1,1-dimethylpropyl, 1-hexyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 2,2-dimethylbutyl, 3,3-dimethylbutyl, 1-ethyl-1-methylpropyl and the like can be mentioned.

The “lower alkenyl group” for R¹ is a linear or branched alkenyl group having 2 to 6 carbon atoms. As specific examples, for example, ethenyl, 1-propenyl, 2-propenyl, 1-butenyl, 2-butenyl, 3-butenyl, 1,3-butadienyl, 1-pentenyl, 2-pentenyl, 3-pentenyl, 4-pentenyl, 1-hexenyl, 2-hexenyl, 3-hexenyl, 4-hexenyl, 5-hexenyl and the like can be mentioned.

The “aryl group” for R¹, for example, a C₆-C₁₀ aryl group such as phenyl, naphthyl, pentalenyl and the like can be mentioned.

The “heterocyclic group” for R¹ is a saturated or unsaturated monocyclic or polycyclic heterocyclic group containing at least one hetero atom such as oxygen atom, sulfur atom, nitrogen atom or selenium atom and the like. As specific examples, for example, an unsaturated 3 to 8-membered (more preferably 5 or 6-membered) heteromonocyclic group containing 1 to 4 nitrogen atom such as pyrrolyl, pyrrolinyl, imidazolyl, pyrazolyl, pyridyl, dihydropyridyl, pyrimidyl, pyrazinyl, pyridazinyl, triazolyl (e.g., 4H-1,2,4-triazolyl, 1H-1,2,3-triazolyl, 2H-1,2,3-triazolyl and the like), tetrazolyl (e.g., 1H-tetrazolyl, 2H-tetrazolyl and the like) and the like; an unsaturated 3 to 8-membered (more preferably 5 or 6-membered) heteromonocycle group containing 1 to 2 sulfur atom such as thienyl, dihydro dithinyl, dihydrodithionyl and the like; an unsaturated 3 to 8-membered (more preferably 5 or 6-membered) heteromonocycle group containing 1 oxygen atom such as furyl and the like, can be mentioned.

As the “aryl group” and “heterocyclic group” for R², those exemplarily recited for R¹ can be mentioned.

As the “halogen atom” for R¹ and R², fluorine atom, chlorine atom, bromine atom, iodine atom and the like can be mentioned. As preferable halogen atom, fluorine atom, chlorine atom, bromine atom can be mentioned.

In the formula (I), A¹ is an optionally substituted divalent group of heterobicycle, or a group represented by —CH═CH-A²- wherein A² is an optionally substituted divalent group of nitrogen-containing 5- or 6-membered heterocycle.

The “optionally substituted divalent group of heterobicycle” is a divalent group of saturated or unsaturated heterobicycle containing at least one hetero atom such as oxygen atom, sulfur atom, nitrogen atom or selenium atom. Of these, a divalent group of benzimidazole, indole, imidazopyridine, benzofuran or indazole is preferable.

The “optionally substituted divalent group of nitrogen-containing 5- or 6-membered heterocycle” for A² is a divalent group of 5- or 6-membered heterocycle containing at least one nitrogen atom. Of these, a divalent group of imidazole is preferable.

The above-mentioned lower alkyl group, lower alkenyl group, aryl group, heterocyclic group, divalent group of heterobicycle and divalent group of nitrogen-containing 5- or 6-membered heterocycle are optionally substituted by at least one group selected from the group consisting of a lower alkyl group, a lower alkenyl group, a lower alkynyl group, a lower alkoxy group, a lower alkylthio group, an amino group, a morpholino group, an acyl group, a halogen atom, an aryl group, a heterocyclic group and a nitro group. These substituents optionally have other substituent at substitutable moiety. As the “lower alkyl group”, “lower alkenyl group”, “halogen atom”, “aryl group” and “heterocyclic group”, those exemplarily recited for R¹ can be mentioned.

The “lower alkynyl group” is a linear or branched alkynyl group having 2 to 6 carbon atoms. As specific examples, for example, ethynyl, 1-propynyl, 2-propynyl, 1-butynyl, 2-butynyl, 3-butynyl, 1-pentynyl, 2-pentynyl, 3-pentynyl, 4-pentynyl, 2-methyl-3-butynyl, 1,1-dimethyl-2-butynyl, 1-hexynyl, 5-hexynyl and the like can be mentioned.

The “lower alkoxy group” is a linear or branched alkyloxy group having up to 6 carbon atoms. As preferable examples, for example, methoxy group, ethoxy group, 1-propoxy group, i-propoxy group, 1-butoxy group, i-butoxy group, sec-butoxy group, tert-butoxy group, 1-pentyloxy group, i-pentyloxy group, sec-pentyloxy group, tert-pentyloxy group, 2-methylbutoxy group, 1-hexyloxy group, i-hexyloxy group, tert-hexyloxy group, sec-hexyloxy group, 2-methylpentyloxy group, 3-methylpentyloxy group, 1-ethylbutoxy group, 2-ethylbutoxy group, 1,1-dimethylbutoxy group, 2,2-dimethylbutoxy group, 3,3-dimethylbutoxy group, and 1-ethyl-1-methylpropoxy group and the like can be mentioned. As alkoxy group having up to 5 carbon atoms, for example, methoxy group, ethoxy group, 1-propoxy group, i-propoxy group, 1-butoxy group, i-butoxy group, sec-butoxy group, tert-butoxy group, 1-pentyloxy group and the like are more preferable.

The “lower alkylthio group” is a linear or branched alkylthio group having up to 6 carbon atoms. As preferable examples, for example, methylthio group, ethylthio group, n-propylthio group, i-propylthio group, n-butylthio group, i-butylthio group, sec-butylthio group, tert-butylthio group, n-pentylthio group, i-pentylthio group, sec-pentylthio group, tert-pentylthio group, 2-methylbutylthio group, n-hexylthio group, i-hexylthio group, t-hexylthio group, sec-hexylthio group, 2-methylpentylthio group, 3-methylpentylthio group, 1-ethylbutylthio group, 2-ethylbutylthio group, 1,1-dimethylbutylthio group, 2,2-dimethylbutylthio group, 3,3-dimethylbutylthio group, and 1-ethyl-1-methylpropylthio group and the like can be mentioned. As alkylthio group having up to 4 carbon atoms, for example, methylthio group, ethylthio group, n-propylthio group, i-propylthio group, n-butylthio group, i-butylthio group, sec-butylthio group, tert-butylthio group and the like are more preferable.

As the “acyl group”, for example, formyl; a lower alkylcarbonyl group which alkyl moiety has up to 6 carbon atoms (e.g., acetyl, propionyl and the like); a lower alkoxycarbonyl group which alkoxy moiety has up to 6 carbon atoms (e.g., n-propoxycarbonyl, i-propoxycarbonyl and the like) and the like can be mentioned. Of these an acyl group such as formyl, acetyl, propionyl, n-propoxycarbonyl, i-propoxycarbonyl and the like, wherein the number of carbon atoms other than that of the carbonyl moiety is up to 3, are preferable.

Preferably R¹ is a lower alkyl group; a lower alkenyl group optionally substituted by aryl group; an aryl group optionally substituted by lower alkyl group or lower alkenyl group; or a heterocyclic group optionally substituted by halogen atom. More preferably R¹ is a lower alkyl group, a lower alkenyl group optionally substituted by phenyl group, a phenyl group optionally substituted by lower alkyl group or lower alkenyl group, or a halothienyl group, still more preferably, butyl group, pentyl group, benzyl group, phenyl group, phenylbenzyl group, 5-chloro-2-thienyl group, 5-bromo-2-thienyl group, pentenyl group, phenylvinyl group, vinylphenyl group.

The “halothienyl group” is a thienyl group substituted by at least one halogen atom mentioned above. As preferable examples, for example, chlorothienyl, bromothienyl and the like can be mentioned.

Preferably R² is an aryl group or heterocyclic group substituted by at least one group selected from the group consisting of an optionally substituted lower alkyl group, an optionally substituted lower alkenyl group, an optionally substituted lower alkynyl group, an optionally substituted lower alkoxy group, a lower alkylthio group, an optionally substituted amino group, a morpholino group, an acyl group, a halogen atom, an aryl group, an optionally substituted heterocyclic group and a nitro group. More preferably, R² is a group selected from the group consisting of phenyl group, naphthyl group and pyridyl group, which is substituted by at least one group selected from the group consisting of a lower alkyl group optionally substituted by a halogen atom, a phenyl group, a phenoxy group or a cyclo(lower)alkyloxy group; a lower alkenyl group; a lower alkynyl group optionally substituted by phenyl group; a lower alkoxy group optionally substituted by phenyl group, cyclo(lower)alkyl group or thienyl group; an lower alkylthio group; a lower alkylamino group optionally substituted by lower alkoxycarbonyl group; a lower alkanoylamino group; a lower alkoxycarbonyl group; a halogen atom; a phenyl group; a thienyl group optionally substituted by halogen atom; a furyl group; a morpholino group; and a nitro group.

The “cyclo(lower)alkyl group” is a cyclic lower alkyl group having up to 6 carbon atoms. As preferable examples, for example, cyclopropyl group, cyclobutyl group, cyclopentyl group, cyclohexyl group and the like can be mentioned.

The “cyclo(lower)alkyloxy group” is an oxy group substituted by a cyclic lower alkyl group having up to 6 carbon atoms. As preferable examples, for example, cyclopropyloxy group, cyclobutyloxy group, cyclopentyloxy group, cyclohexyloxy group and the like can be mentioned.

The “lower alkylamino group” is a linear and branched alkylamino group having up to 6 carbon atoms. As preferable examples, for example, methylamino group, ethylamino group, n-propylamino group, i-propylamino group, n-butylamino group, i-butylamino group, sec-butylamino group, tert-butylamino group, n-pentylamino group, i-pentylamino group, sec-pentylamino group, tert-pentylamino group, 2-methylbutylamino group, n-hexylamino group, i-hexylamino group, t-hexylamino group, sec-hexylamino group, 2-methylpentylamino group, 3-methylpentylamino group, 1-ethylbutylamino group, 2-ethylbutylamino group, 1,1-dimethylbutylamino group, 2,2-dimethylbutylamino group, 3,3-dimethylbutylamino group, and 1-ethyl-1-methylpropylamino group and the like can be mentioned. More preferably, an alkylamino group having up to 4 carbon atoms, for example, methylamino group, ethylamino group, n-propylamino group, i-propylamino group, n-butylamino group, i-butylamino group, sec-butylamino group, tert-butylamino group and the like can be mentioned.

The “lower alkanoylamino group” is a linear and branched alkanoylamino group having up to 6 carbon atoms. As preferable examples, for example, methylcarbonylamino group, ethylcarbonylamino group, n-propylcarbonylamino group, i-propylcarbonylamino group, n-butylcarbonylamino group, i-butylcarbonylamino group, sec-butylcarbonylamino group, tert-butylcarbonylamino group and the like can be mentioned.

The definitions of other groups are as mentioned above.

Still more preferably, R² is 2,4-dichlorophenyl, 3-chloro-4-biphenylyl, 1-bromo-2-naphthyl, 2-chloro-4-(phenoxymethyl)phenyl, 2-chloro-4-ethylphenyl, 2-chloro-4-(2-thienyl)phenyl, 2-chloro-4-nitrophenyl, 2-chloro-4-(trifluoromethyl)phenyl, 4-(benzyloxy)phenyl, 2-chloro-4-(2-phenylethyl)phenyl, 2-chloro-4-(methylthio)phenyl, 2-chloro-4-(pentylthio)phenyl, 2-chloro-4-(pentylamino)phenyl, 2-chloro-4-(2-thienylmethoxy)phenyl, 2-chloro-4-(4-morpholino)phenyl, 2-chloro-4-(i-propoxycarbonyl)phenyl, 2-chloro-4-ethoxyphenyl, 2-chloro-4-(1-hexyn-1-yl)phenyl, 4-bromo-2-chlorophenyl, 4-benzyloxy-2-chlorophenyl, 2-chloro-4-(cyclohexylmethoxy)phenyl, 2-chloro-4-(phenylethynyl)phenyl, 2-chloro-4-(1-hexen-1-yl)phenyl, 2-chloro-4-[(cyclohexyloxy)methyl]phenyl, 2-chloro-4-(2-thienyl)phenyl, 2-chloro-4-(2-furyl)phenyl, 2-chloro-4-(3-methylbutoxy)phenyl, 2-chloro-4-[methyl(pentyl)amino]phenyl, 2-chloro-4-(cyclopentylmethoxy)phenyl, 2-chloro-4-[methyl(ethoxycarbonyl)amino]phenyl, 2-methyl-4-[ethyl(pentyl)amino]phenyl, 2-chloro-4-(butylcarbonylamino)phenyl, 3-chloro-5-trifluoromethyl-2-pyridinyl, 2-chloro-6-phenyl-3-pyridinyl, 2-chloro-4-pentyloxyphenyl, 2-chloro-4-hexenylphenyl, 2-chloro-4-(1-pentyn-1-yl)phenyl or 2-chloro-4-propoxyphenyl.

Preferably, A¹ is a divalent group of heterobicycle selected from the group consisting of benzimidazole, indole, imidazopyridine, benzofuran and indazole, which is substituted by at least one lower alkyl group; or A¹ is a group represented by —CH═CH-A²- wherein A² is a divalent group of imidazole substituted by at least one group selected from the group consisting of a lower alkyl group and a halogen atom. More preferably, A¹ is a divalent group derived from 2-methyl-1H-benzimidazole, 2,4-dimethyl-1H-benzimidazole, 2-methyl-1H-indole, 2-methyl-3H-imidazo[4,5-b]pyridine, 2,7-dimethyl-3H-imidazo[4,5-b]pyridine, 2-methylbenzofuran, 3-methyl-1H-indazole, 4-chloro-2-methyl-1H-imidazol-5-ylvinyl or 4-chloro-2-ethyl-1H-imidazol-5-ylvinyl.

The sulfonamide compound to be used in the present invention may form a salt, and when used as a pharmaceutical product, a pharmaceutically acceptable salt is preferable. As the pharmaceutically acceptable salt, for example, salts with inorganic acid, such as hydrochloric acid, sulfuric acid, phosphoric acid, diphosphoric acid, hydrobromic acid and nitric acid and the like, salts with organic acid, such as acetic acid, malic acid, maleic acid, fumaric acid, tartaric acid, succinic acid, citric acid, lactic acid, methanesulfonic acid, p-toluenesulfonic acid, palmitic acid, salicylic acid and stearic acid and the like, salts with inorganic base such as alkali metal (e.g., sodium, potassium etc.), alkaline earth metal (e.g., calcium, magnesium etc.), ammonium and the like, salts with organic base such as triethylamine, diisopropylethylamine, pyridine, picoline, ethanolamine, triethanolamine, dicyclohexylamine, N,N′-dibenzylethylenediamine and the like, salts with basic or acidic amino acid such as arginine, aspartic acid, glutamic acid and the like can be mentioned.

The compounds represented by the formula (I) and a pharmaceutically acceptable salt thereof can be produced by, for example, the methods described in the above-mentioned WO97/24334, WO98/15530, WO99/00359, WO99/00372, WO99/00373, WO99/51574, WO00/34277, WO00/39097, WO00/39099 and the like or a method analogous thereto.

As preferable examples of the sulfonamide compound to be used in the present invention, the following compounds can be mentioned.
(1) 3-[(3-chloro-4-biphenylyl)methyl]-2-methyl-N-(pentylsulfonyl)-1H-indole-5-carboxamide
(2) 3-[(1-bromo-2-naphthyl)methyl]-2-methyl-N-[(4-methylphenyl)sulfonyl]-1H-indole-5-carboxamide
(3) 1-(2,4-dichlorobenzyl)-2-methyl-N-(pentylsulfonyl)-1H-benzimidazole-6-carboxamide
(4) N-(butylsulfonyl)-1-(2,4-dichlorobenzyl)-2,4-dimethyl-1H-benzimidazole-6-carboxamide
(5) N-(n-pentanesulfonyl)-4-amino-3-(2,4-dichlorobenzylamino)benzamide
(6) (E)-3-(4-bromo-1-(2,4-dichlorobenzyl)-2-methylimidazol-5-yl)-N-(n-pentanesulfonyl)-2-propenamide
(7) (E)-N-(n-pentanesulfonyl)-2-(4-phenylphenyl)ethenylpyridine-4-carboxamide
(8) 3-[2-chloro-4-(phenoxymethyl)benzyl]-2-methyl-N-(phenylsulfonyl)-3H-imidazo[4,5-b]pyridine-5-carboxamide
(9) 3-(2-chloro-4-ethylbenzyl)-2-methyl-N-[(4-methylphenyl)sulfonyl]-3H-imidazo[4,5-b]pyridine-5-carboxamide
(10) 3-[2-chloro-4-(5-chloro-2-thienyl)benzyl]-2-methyl-N-(phenylsulfonyl)-3H-imidazo[4,5-b]pyridine-5-carboxamide
(11) 3-(2-chloro-4-nitrobenzyl)-2,7-dimethyl-N-[(4-methylphenyl)sulfonyl]-3H-imidazo[4,5-b]pyridine-5-carboxamide
(12) 3-[(1-bromo-2-naphthyl)methyl]-N-[(5-chloro-2-thienyl)sulfonyl]-2,7-dimethyl-3H-imidazo[4,5-b]pyridine-5-carboxamide
(13) N-[(5-bromo-2-thienyl)sulfonyl]-3-[2-chloro-4-(trifluoromethyl)benzyl]-2-methyl-3H-imidazo[4,5-b]pyridine-5-carboxamide
(14) 3-[(3-chloro-4-biphenylyl)methyl]-2-methyl-N-(4-penten-1-ylsulfonyl)-3H-imidazo[4,5-b]pyridine-5-carboxamide
(15) 3-[4-(benzyloxy)-2-chlorobenzyl]-N-[(5-chloro-2-thienyl)sulfonyl]-2-methyl-3H-imidazo[4,5-b]pyridine-5-carboxamide
(16) N-[(5-bromo-2-thienyl)sulfonyl]-3-[2-chloro-4-(2-phenylethyl)benzyl]-2-methyl-3H-imidazo[4,5-b]pyridine-5-carboxamide
(17) N-[(5-bromo-2-thienyl)sulfonyl]-3-[2-chloro-4-(methylthio)benzyl]-2-methyl-3H-imidazo[4,5-b]pyridine-5-carboxamide
(18) 3-[2-chloro-4-(pentylthio)benzyl]-2-methyl-N-[(4-methylphenyl)sulfonyl]-3H-imidazo[4,5-b]pyridine-5-carboxamide
(19) 3-[2-chloro-4-(pentylamino)benzyl]-2-methyl-N-[(4-methylphenyl)sulfonyl]-3H-imidazo[4,5-b]pyridine-5-carboxamide
(20) 3-[2-chloro-4-(2-thienylmethoxy)benzyl]-2-methyl-N-[(4-methylphenyl)sulfonyl]-3H-imidazo[4,5-b]pyridine-5-carboxamide
(21) 3-[2-chloro-4-(4-morpholino)benzyl]-2,7-dimethyl-N-[(4-methylphenyl)sulfonyl]-3H-imidazo[4,5-b]pyridine-5-carboxamide
(22) isopropyl 3-chloro-4-{[2-methyl-5-({[(4-methylphenyl)sulfonyl]amino}carbonyl)-3H-imidazo[4,5-b]pyridin-3-yl]methyl}benzoate
(23) 3-(2-chloro-4-ethylbenzyl)-2,7-dimethyl-N-[(4-methylphenyl)sulfonyl]-3H-imidazo[4,5-b]pyridine-5-carboxamide
(24) 3-(2-chloro-4-ethoxybenzyl)-2,7-dimethyl-N-[(4-methylphenyl)sulfonyl]-3H-imidazo[4,5-b]pyridine-5-carboxamide
(25) 3-[2-chloro-4-(1-hexyn-1-yl)benzyl]-2-methyl-N-[(4-methylphenyl)sulfonyl]-3H-imidazo[4,5-b]pyridine-5-carboxamide
(26) 3-[(3-chloro-4-biphenylyl)methyl]-2-methyl-N-[(1E)-1-penten-1-ylsulfonyl]-1-benzofuran-5-carboxamide
(27) 3-[(3-chloro-4-biphenylyl)methyl]-2-methyl-N-{[(E)-2-phenylvinyl]sulfonyl}-1-benzofuran-5-carboxamide
(28) 1-[(3-chloro-4-biphenylyl)methyl]-3-methyl-N-[(1E)-1-penten-1-ylsulfonyl]-1H-indazole-6-carboxamide
(29) 1-(2,4-dichlorobenzyl)-2-methyl-N-[(1E)-1-penten-1-ylsulfonyl]-1H-benzimidazole-6-carboxamide
(30) 1-(2,4-dichlorobenzyl)-2-methyl-N-{[(E)-2-phenylvinyl]sulfonyl}-1H-benzimidazole-6-carboxamide
(31) 1-[(3-chloro-4-biphenylyl)methyl]-2-methyl-N-(pentylsulfonyl)-1H-benzimidazole-6-carboxamide (32)1-[2-chloro-4-(trifluoromethyl)benzyl]-2-methyl-N-(pentylsulfonyl)-1H-benzimidazole-6-carboxamide
(33) 1-[(3-chloro-4-biphenylyl)methyl]-2-methyl-N-[(4-methylphenyl)sulfonyl]-1H-benzimidazole-6-carboxamide
(34) 1-[(3-chloro-4-biphenylyl)methyl]-2-methyl-N-{[(E)-2-phenylvinyl]sulfonyl}-1H-benzimidazole-6-carboxamide
(35) 1-(4-bromo-2-chlorobenzyl)-2-methyl-N-[(1E)-1-penten-1-ylsulfonyl]-1H-benzimidazole-6-carboxamide (36)1-[4-(benzyloxy)-2-chlorobenzyl]-2-methyl-N-(pentylsulfonyl)-1H-benzimidazole-6-carboxamide
(37) 1-[2-chloro-4-(cyclohexylmethoxy)benzyl]-2-methyl-N-(pentylsulfonyl)-1H-benzimidazole-6-carboxamide
(38) 1-[2-chloro-4-(cyclohexylmethoxy)benzyl]-2-methyl-N-[(4-methylphenyl)sulfonyl]-1H-benzimidazole-6-carboxamide
(39) 1-(2,4-dichlorobenzyl)-2-methyl-N-[(4-methylphenyl)sulfonyl]-1H-benzimidazole-6-carboxamide
(40) 1-[2-chloro-4-(phenylethynyl)benzyl]-2-methyl-N-(pentylsulfonyl)-1H-benzimidazole-6-carboxamide
(41) 1-{2-chloro-4-[(1E)-1-hexen-1-yl]benzyl}-2-methyl-N-(pentylsulfonyl)-1H-benzimidazole-6-carboxamide
(42) 1-{2-chloro-4-[(1E)-1-hexen-1-yl]benzyl}-2-methyl-N-[(4-methylphenyl)sulfonyl]-1H-benzimidazole-6-carboxamide
(43) 1-{2-chloro-4-[(cyclohexyloxy)methyl]benzyl}-2-methyl-N-(pentylsulfonyl)-1H-benzimidazole-6-carboxamide
(44) 1-[2-chloro-4-(2-thienyl)benzyl]-2-methyl-N-(pentylsulfonyl)-1H-benzimidazole-6-carboxamide
(45) 1-[2-chloro-4-(2-furyl)benzyl]-2-methyl-N-(pentylsulfonyl)-1H-benzimidazole-6-carboxamide
(46) 1-[2-chloro-4-(phenylethynyl)benzyl]-2-methyl-N-[(4-vinylphenyl)sulfonyl]-1H-benzimidazole-6-carboxamide
(47) 1-[2-chloro-4-(3-methylbutoxy)benzyl]-2-methyl-N-[(4-methylphenyl)sulfonyl]-1H-benzimidazole-6-carboxamide
(48) 3-(4-bromo-2-chlorobenzyl)-2-methyl-N-{[(E)-2-phenylvinyl]sulfonyl}-1H-indole-5-carboxamide
(49) 3-[2-chloro-4-(cyclohexylmethoxy)benzyl]-2-methyl-N-[(4-methylphenyl)sulfonyl]-1H-indole-5-carboxamide
(50) 3-[2-chloro-4-(trifluoromethyl)benzyl]-2-methyl-N-[(4-methylphenyl)sulfonyl]-1H-indole-5-carboxamide
(51) 3-[2-chloro-4-(phenoxymethyl)benzyl]-2-methyl-N-(pentylsulfonyl)-1H-indole-5-carboxamide
(52) 3-(2-chloro-4-ethoxybenzyl)-2-methyl-N-(pentylsulfonyl)-1H-indole-5-carboxamide
(53) 3-[2-chloro-4-(2-thienyl)benzyl]-2-methyl-N-[(4-methylphenyl)sulfonyl]-1H-indole-5-carboxamide (54)₃-{2-chloro-4-[methyl(pentyl)amino]benzyl}-2-methyl-N-(pentylsulfonyl)-3H-imidazo[4,5-b]pyridine-5-carboxamide
(55) N-(butylsulfonyl)-3-[2-chloro-4-(cyclopentylmethoxy)benzyl]-2-methyl-3H-imidazo[4,5-b]pyridine-5-carboxamide
(56) ethyl {3-chloro-4-[(2-methyl-5-{[(pentylsulfonyl)amino]carbonyl}-3H-imidazo[4,5-b]pyridin-3-yl)methyl]phenyl}methylcarbamate
(57) 3-{2-chloro-4-[ethyl(pentyl)amino]benzyl}-2-methyl-N-[(4-methylphenyl)sulfonyl]-3H-imidazo[4,5-b]pyridine-5-carboxamide
(58) 3-[2-chloro-4-(pentanoylamino)benzyl]-2-methyl-N-[(4-methylphenyl)sulfonyl]-3H-imidazo[4,5-b]pyridine-5-carboxamide
(59)₃-{[3-chloro-5-(trifluoromethyl)-2-pyridinyl]methyl}-2-methyl-N-[(4-methylphenyl)sulfonyl]-3H-imidazo[4,5-b]pyridine-5-carboxamide
(60)₃-[(2-chloro-6-phenyl-3-pyridinyl)methyl]-2-methyl-N-(pentylsulfonyl)-3H-imidazo[4,5-b]pyridine-5-carboxamide
(61)(2E)-3-{4-chloro-1-[2-chloro-4-(phenylethynyl)benzyl]-2-methyl-1H-imidazol-5-yl}-N-[(4-methylphenyl)sulfonyl]acrylamide
(62)(2E)-3-{4-chloro-1-[2-chloro-4-(pentyloxy)benzyl]-2-ethyl-1H-imidazol-5-yl}-N-{[(E)-2-phenylvinyl]sulfonyl}acrylamide
(63)(2E)-3-{4-chloro-1-[2-chloro-4-(phenylethynyl)benzyl]-2-methyl-1H-imidazol-5-yl}-N-(pentylsulfonyl)acrylamide
(64)(2E)-3-{4-chloro-1-[2-chloro-4-(phenylethynyl)benzyl]-2-ethyl-1H-imidazol-5-yl}-N-[(1E)-1-penten-1-ylsulfonyl]acrylamide sodium salt
(65)(2E)-3-{4-chloro-1-[2-chloro-4-(phenylethynyl)benzyl]-2-ethyl-1H-imidazol-5-yl}-N-[(1E)-1-penten-1-ylsulfonyl]acrylamide
(66) 1-(2-chloro-4-hexylbenzyl)-2-methyl-N-(pentylsulfonyl)-1H-benzimidazole-6-carboxamide
(67) 1-[2-chloro-4-(methylthio)benzyl]-2-methyl-N-(pentylsulfonyl)-1H-benzimidazole-6-carboxamide
(68) 1-[2-chloro-4-(1-pentyn-1-yl)benzyl]-2-methyl-N-(pentylsulfonyl)-1H-benzimidazole-6-carboxamide
(69) N-(butylsulfonyl)-1-[2-chloro-4-(2-furyl)benzyl]-2-methyl-1H-benzimidazole-6-carboxamide
(70) 1-[2-chloro-4-(2-furyl)benzyl]-2-methyl-N-[(1E)-1-penten-1-ylsulfonyl]-1H-benzimidazole-6-carboxamide
(71) 1-(2-chloro-4-propoxybenzyl)-2-methyl-N-(pentylsulfonyl)-1H-benzimidazole-6-carboxamide
(72) 1-[4-(benzyloxy)-2-chlorobenzyl]-2-methyl-N-{[(E)-2-phenylvinyl]sulfonyl}-1H-benzimidazole-6-carboxamide

Of these compounds, the compounds (1), (3), (12), (27), (31), (35), (56), (64) and (65) are particularly preferable.

The compounds of the above-mentioned (1) and (2) can be produced based on the description of Example of WO98/15530. The compounds of the above-mentioned (3) and (4) can be produced based on the description of Example of WO97/24334. The compounds of the above-mentioned (5)-(7) can be produced based on the description of Example of WO99/00359. The compounds of the above-mentioned (8)-(28) can be produced based on the description of Example of WO99/00372. The compounds of the above-mentioned (29)-(47) can be produced based on the description of Example of WO99/00373. The compounds of the above-mentioned (48)-(53) can be produced based on the description of Example of WO99/51574. The compounds of the above-mentioned (54)-(60) can be produced based on the description of Example of WO00/34277. The compounds of the above-mentioned (61)-(65) can be produced based on the description of Example of WO00/39097. The compounds of the above-mentioned (66)-(72) can be produced based on the description of Example of WO00/39099.

The sulfonamide compound to be used in the present invention may be a prodrug. A “prodrug” means a compound that changes into a sulfonamide compound to be used in the present invention, preferably a sulfonamide compound represented by the formula (I), more preferably compound (1)-(72), due to the reaction of enzyme, gastric acid and the like under physiological conditions in living organisms, namely, a compound that changes into these compounds as a result of oxidization, reduction, hydrolysis and the like due to enzyme, gastric acid and the like.

In the pharmaceutical agent of the present invention, as a pharmaceutical agent to be combined with a sulfonamide compound, α-glucosidase inhibitors, sulfonylureas, insulin secretagbgues, insulin preparations, biguanides, β-hydroxy-β-methyl glutaryl CoA (HMG-CoA) reductase inhibitors, calcium antagonists, fibrates, diuretic drugs, angiotensin converting enzyme (ACE) inhibitors, angiotensin II antagonists, cholesterol absorption inhibitors, antioxidants, nicotinic acid derivatives, squalene synthesis inhibitors, aldose reductase inhibitors, β3 agonists, peroxisome proliferator-activated receptor (PPAR) regulators, dipeptidyl peptidase 4 (DPP4) inhibitors, glucagon-like peptide-1 (GLP-1) analogues, sodium-dependent glucose transporter (SGLT) inhibitors, 11β-hydroxysteroid dehydrogenase 1 (11β-HSD1) inhibitors, Microsomal triglyceride transfer protein (MTP) inhibitors, acyl-CoA:cholesterol acyltransferase (ACAT) inhibitors, intestinal bile acid transporter (IBAT) inhibitors, ezetimibe, vascular adhesion protein 1 (VAP1) inhibitors, advanced glycation end product (AGE) inhibitors, lipase inhibitors, antifeedants, leptin (hereinafter sometimes to be simply referred to collectively as other therapeutic drug) and the like can be mentioned. Of these, an α-glucosidase inhibitors, an insulin secretagogues, sulfonylureas and biguanides are preferable.

α-glucosidase inhibitors are pharmaceutical agents having an action to inhibit digestive enzymes such as amylase, maltase, α-dextrinase, sucrase and the like to delay digestion of starch and sucrose.

As preferable α-glucosidase inhibitors, miglitol ([2R(2α, 3α, 4α, 5β)]-1-(2-hydroxyethyl)-2-(hydroxymethyl)-3,4,5-piperidinetriol), voglibose (3,4-dideoxy-4-[[2-hydroxy-1-(hydroxymethyl)ethyl]amino]-2-C-(hydroxymethyl)-D-epi-inositol), miglustat (N-butyl-1-deoxynojirimycin), acarbose (0-4,6-dideoxy-4-[[1S-(1α,4α,5α,6α)-4,5,6-trihydroxy-3-(hydroxymethyl)-2-cyclohexen-1-yl]amino]1-α-D-glucopyranosyl-(1→44)-O-β-D-glucopyranosyl-(1→4)-D-glucose), celgosivir hydrochloride ([1S-(1α, 6β, 7α, 8αβ)]-octahydro-1,7,8-trihydroxy-6-indolizinyl butanoate hydrochloride) and the like can be mentioned.

An insulin secretagogues are pharmaceutical agents having an action to promote insulin secretion from pancreatic β cells. As the insulin secretagogues, for example, sulfonylureas (SU agent) can be mentioned. The sulfonylureas (SU agent) are pharmaceutical agents that transmits insulin secretion signal via an SU receptor in cell membrane to promote insulin secretion from pancreatic β cells.

As preferable insulin secretagogues, nategnide (N-(trans-4-isopropylcyclohexylcarbonyl)-D-phenylalanine), glimepiride (trans-3-ethyl-2,5-dihydro-4-methyl-N-[2-[4-[[[[(4-methylcyclohexyl)amino]carbonyl]amino]sulfonyl]phenyl]ethyl]-2-oxo-1H-pyrrole-1-carboxamide), repaglinide ((+)-2-ethoxy-α-[[(S)-α-isobutyl-o-piperidinobenzyl]carbamoyl]-p-toluic acid), glisentide (1-cyclopentyl-3-p-(2-O-anisamidoethyl)benzenesulfonylurea), mitiglinide (calcium (−)-2(S)-benzyl-4-oxo-4-(cis-perhydroisoindol-2-yl)butyrate dihydrate), glucagons-like peptide-17-36-amide, glucagons-like peptide-1-amylin, CJC1131 and the like can be mentioned.

As other insulin secretion inhibitors, for example, N-[[4-(1-methylethyl)cyclohexyl]carbonyl]-D-phenylalanine(AY-4166), calcium (2S)-2-benzyl-3-(cis-hexahydro-2-isoindolinylcarbonyl)propionate dihydrate (KAD-1229), glimepiride (Hoe490) and the like can be mentioned.

As preferable sulfonylureas, glimepiride (trans-3-ethyl-2,5-dihydro-4-methyl-N-[2-[4-[[[[(4-methylcyclohexyl)amino]carbonyl]amino]sulfonyl]phenyl]ethyl]-2-oxo-1H-pyrrole-1-carboxamide), glisentide (1-cyclopentyl-3-p-(2-o-anisamidoethyl)benzenesulfonyl]urea) and the like can be mentioned.

As other sulfonylureas, for example, tolbutamide, chlorpropamide, tolazamide, acetohexamide, 4-chloro-N-[(1-pyrrolidinylamino)carbonyl]-benzenesulfonamide (general name: glyclopyramide) and an ammonium salt thereof, glibenclamide (glyburide), gliclazide, 1-butyl-3-metanilylurea, carbutamide, glibonuride, glipizide, gliquidone, glisoxepid, glybuthiazole, glybuzole, glyhexamide, glymidine, glypinamide, phenbutamide, and tolylcyclamide and the like can be mentioned.

Biguanides are pharmaceutical agents having actions of stimulation of anaerobic glycolysis, increase of the sensitivity to insulin in the peripheral tissues, inhibition of glucose absorption from the intestine, suppression of hepatic gluconeogenesis, and inhibition of fatty acid oxidation.

As preferable biguanides, phenformin (1-phenethylbiguanide), metformin (1,1-dimethylbiguanide), buformin (1-butylbiguanide) and the like can be mentioned.

As examples of the HMG-CoA reductase inhibitors, rosvastatin calcium, atorvastatin calcium hydrate, pitavastatin calcium, fluvastatin sodium, simvastatin, lovastatin, pravastatin sodium and the like can be mentioned.

As the calcium antagonist, aranidipine, lacidipine, naftopidil, felodipine, azelnidipine, cilnidipine, lomerizine, diltiazem, gallopamil, efonidipine, nisoldipine, amlodipine, lercanidipine, bevantolol, nicardipine, isradipine, benidipine, verapamil, nitrendipine, barnidipine, propafenone, manidipine, bepridil, nifedipine, nilvadipine, nimodipine, fasudil, pirmenol, carvedilol, trimetazidine, ethosuximide, zonisamide, felodipine, propiverine, manidipine, temiverine, ziconotide and the like can be mentioned.

As the fibrates, gemfibrozil, fenofibrate, bezafibrate, ciplofibrate, clinofibrate, clofibrate and the like can be mentioned.

As the diuretic drugs, cicletanine hydrochloride, torasemide, tripamide, potassium canrenoate, isosorbide, piretanide, azosemide, indapamide, hydrochlorothiazide, trichlormethiazide, benzylhydrochlorothiazide, meticrane, chlorthalidone, mefruside, furosemide, spironolactone, triamterene and the like can be mentioned.

As the ACE inhibitors, trandolapril, moexipril, perindopril, quinapril hydrochloride, spirapril hydrochloride, temocapril, cilazapril, fosinopril, zofenopril, imidapril, quinapril, benazepril hydrochloride, lisinopril, captopril, ramipril, delapril, alacepril, enalapril, omapatrilat and the like can be mentioned.

As the angiotensin II antagonists, candesartan cilexetil, irbesartan, ormesartan medoxomil, termisartan, valsartan, eprosartan, losartan potassium and the like can be mentioned.

As the cholesterol absorption inhibitors, colesevelam, ezetimibe, colestimide, colestyramine, ion exchange resinpreparation and the like can be mentioned.

As the antioxidants, probucol, vitamin E and the like can be mentioned.

As the nicotinic acid derivatives, tocopherol nicotinate, nicomol, niceritrol and the like can be mentioned.

As the squalene synthesis inhibitors, TAK-475, YM-53601 and the like can be mentioned.

As the aldose reductase inhibitors, epalrestat, zenarestat, IDD-598, NZ-314, AS-3201 and the like can be mentioned.

As the PPAR regulators, thiazolidinedione antidiabetic agents such as rosiglitazone, pioglitazone, troglidazone, EML-16336 and the like, and the like can be mentioned.

As the β3 agonists, GRC-1087, YM-178, SR58611A, L 796568 and the like can be mentioned.

As the ACAT inhibitors, melinamide, eflucimibe, pactimibe, and the like can be mentioned.

As the lipase inhibitors, docosanol, orlistat and the like can be mentioned.

As the antifeedants, mazindol and the like can be mentioned.

Using the pharmaceutical agent of the present invention, diseases such as impaired glucose tolerance disorder, diabetes (e.g., type II diabetes), gestational diabetes, diabetic complications (e.g., diabetic gangrene, diabetic arthropathy, diabetic osteopenia, diabetic glomerulosclerosis, diabetic nephropathy, diabetic dermatopathy, diabetic neuropathy, diabetic cataract, diabetic retinopathy and the like), insulin resistance syndrome (e.g., insulin receptor abnormality, Rabson-Mendenhall syndrome, leprechaunism, Kobberling-Dunnigan syndrome, Lawrence-Seip syndrome (adipose tissue atrophy), Cushing syndrome, acromegaly and the like), polycystic ovary syndrome, hyperlipidemia, atherosclerosis, cardiovascular diseases (e.g., stenocardia, cardiac failure and the like), hyperglycemia (e.g., those characterized by abnormal saccharometabolism such as eating disorders), pancreatitis, osteoporosis, hyperuricemia, hypertension, inflammatory bowel diseases, and skin disorders related to an anomaly of differentiation of epidermic cells and the like in mammals (e.g., mouse, rat, hamster, rabbit, cat, dog, bovine, sheep, monkey, human and the like) can be effectively treated and prevented.

The pharmaceutical agent of the present invention comprises a combination of a sulfonamide compound and other therapeutic agent, which is a concomitant agent.

The “pharmaceutical agent comprising a combination of a sulfonamide compound and other therapeutic agent” includes both a single preparation comprising a sulfonamide compound and other therapeutic agent and a preparation comprising a combination of separate preparations of a sulfonamide compound and other therapeutic agent (e.g., commercial package, kit etc.).

The mode of administration is not particularly limited, and, for example, (1) administration of a composition comprising a sulfonamide compound and an other therapeutic agent, i.e., a single preparation, (2) simultaneous administration of two kinds of preparations obtained by separately processing the sulfonamide compound and the other therapeutic agent by the same administration route, (3) time staggered administration of two kinds of preparations obtained by separately processing a sulfonamide compound and an other therapeutic agent by the same administration route (e.g., administration in the order of a sulfonamide compound and an other therapeutic agent, or administration in reverse order), (4) simultaneous administration of two kinds of preparations obtained by separately processing a sulfonamide compound and an other therapeutic agent by different administration routes, and (5) time staggered administration of two kinds of preparations obtained by separately preparing a sulfonamide compound and an other therapeutic agent by different administration routes (e.g., administration in the order of a sulfonamide compound and an other therapeutic agent, or administration in reverse order) and the like can be mentioned.

As the pharmaceutical agent of the present invention, sulfonamide compound and other therapeutic agent can be administered orally or parenterally, separately or simultaneously, as it is or upon admixing with a pharmacologically acceptable carrier and the like, in the form of, for example, a solid preparation such as powder, granule, tablet, capsule and the like, a liquid such as syrup, emulsion, injection (including subcutaneous injection, intravenous injection, intramuscular injection, intravenous infusion) and the like, sublingual tablet, buccal, troche, microcapsule, a preparation with a sustained release coating and the like, or a suppository.

As the above-mentioned pharmacologically acceptable carrier, there are mentioned various conventional organic or inorganic carriers as a material for the preparation. Examples thereof include excipients, lubricants, binders and disintegrators for solid preparations; and solvents, solubilizing aids, suspending agents, isotonic agents, buffers and soothing agents for liquid preparations. Where necessary, conventional additives such as antiseptics, antioxidants, coloring agents, sweeteners, fragrance and the like can be used.

As preferable examples of the excipient, there are mentioned, for example, lactose, sucrose, D-mannitol, starch, crystalline cellulose, light anhydrous silicic acid, calcium carbonate, calcium phosphate and the like.

As preferable examples of the lubricant, there are mentioned, for example, stearic acid, magnesium stearate, calcium stearate, talc, colloidal silica and the like.

As preferable examples of the binder, there are mentioned, for example, crystalline cellulose, sucrose, D-mannitol, dextrin, hydroxypropylcellulose, hydroxypropylmethylcellulose, polyvinylpyrrolidone, acacia, gelatin and the like.

As preferable examples of the disintegrator, there are mentioned, for example, starch, carboxymethylcellulose, carboxymethylcellulose calcium, croscarmellose sodium, sodium carboxymethyl starch and the like.

As preferable examples of the solvent, there are mentioned, for example, injectable water, physiological saline, alcohol, propylene glycol, Macrogol, sesame oil, corn oil and the like.

As preferable examples of the solubilizing aid, there are mentioned, for example, polyethylene glycol, propylene glycol, D-mannitol, benzyl benzoate, ethanol, tris-aminomethane, cholesterol, triethanolamine, sodium carbonate, sodium citrate and the like.

As preferable examples of the suspending agent, there are mentioned, for example, surfactants such as stearyl triethanolamine, sodium lauryl sulfate, lauryl aminopropionic acid, lecithin, benzalkonium chloride, benzethonium chloride, glyceryl monostearate and the like; hydrophilic polymers such as polyvinyl alcohol, polyvinylpyrrolidone, sodium carboxymethylcellulose, methylcellulose, hydroxymethylcellulose, hydroxyethylcellulose, hydroxypropylcellulose and the like.

As preferable examples of the isotonicity agent, there are mentioned, for example, sodium chloride, glycerine, D-mannitol and the like.

As preferable examples of the buffer, there are mentioned, for example, buffers such as phosphate, acetate, carbonate, citrate and the like.

As preferable examples of the soothing agent, there are mentioned, for example, benzyl alcohol and the like.

As preferable examples of the antiseptic, there are mentioned, for example, p-oxybenzoates, chlorobutanol, benzyl alcohol, phenethyl alcohol, dehydroacetic acid, sorbic acid and the like.

As preferable examples of the antioxidant, there are mentioned, for example, sulfite, ascorbic acid and the like.

As preferable examples of the coloring agent, there are mentioned, for example, natural dye, synthetic dye and the like.

As preferable examples of the sweetening agent, there are mentioned, for example, lactose, sucrose and the like.

A preparation having the above-mentioned dosage form can be produced according to a formulation method known in the pertinent field.

The daily dose of the pharmaceutical agent of the present invention varies depending on the degree of condition, age, sex, body weight and sensitivity to the pharmaceutical agent of the subject of administration, administration period, administration intervals, administration route, kind of pharmaceutical agent to be used concurrently and the like, and is not particularly limited. The dose of a sulfonamide compound is not particularly limited as long as the side effects do not pose any problem, but generally, it is about 0.001-100 mg, preferably about 0.01-2.0 mg, more preferably about 0.05-0.5 mg, per 1 kg body weight of mammal by oral administration. The dose of the pharmaceutical agent to be used concurrently is not particularly limited as long as the side effects pose no problems. For example, when α-glucosidase inhibitors are used concurrently, the daily dose thereof is generally about 0.01-100 mg, preferably about 0.1-50 mg, more preferably about 0.5-10 mg, per 1 kg body weight of mammal by oral administration. When insulin secretagogues are used concurrently, the daily dose thereof is generally about 0.01-100 mg, preferably about 0.1-50 mg, more preferably about 0.5-10 mg, per 1 kg body weight of mammal by oral administration. When sulfonylureas are used concurrently, the daily dose thereof is generally about 0.01-100 mg, preferably about 0.1-50 mg, more preferably about 0.5-10 mg, per 1 kg body weight of mammal by oral administration. When biguanides are used concurrently, the daily dose thereof is generally about 0.1-500 mg, preferably about 1-100 mg, more preferably about 10-30 mg, per 1 kg body weight of mammal by oral administration.

### EXAMPLES

### Example 1

Each of the test compound of the aforementioned (1), (3), (56) or (64) and/or an α-glucosidase inhibitor (e.g., voglibose and the like) or a combination of the both pharmaceutical agents was repeatedly administered to Zucker fatty rats, after which blood glucose level was measured. The results are expressed in the average+standard error of each group. Differences between groups were examined for statistical significance. P<0.05 was considered statistically significant.

The blood glucose level showed greater decrease by the combined administration of the test compound and the α-glucosidase inhibitor than by the single administration of the test compound or the α-glucosidase inhibitor.

### Example 2

Each of the test compound of the aforementioned (1), (3), (56) or (64) and/or an insulin secretagogue (e.g., glibenclamide and the like) or a combination of the both pharmaceutical agents was repeatedly administered to Zucker fatty rats, after which an oral glucose tolerance test was performed. The results are expressed in the average+standard error of each group. Differences between groups were examined for statistical significance. P<0.05 was considered statistically significant.

Increase in the blood glucose level after glucose tolerance was more strongly suppressed by the combined administration of the test compound and the insulin secretagogue than by the single administration of the test compound or the insulin secretagogue.

### Example 3

Each of the test compound of the aforementioned (1), (3), (56) or (64) and/or a biguanide (e.g., metformin and the like) or a combination of the both pharmaceutical agents was repeatedly administered to Zucker fatty rats, after which blood glucose level was measured. The results are expressed in the average±standard error of each group. Differences between groups were examined for statistical significance. P<0.05 was considered statistically significant.

The blood glucose level showed greater decrease by the combined administration of the test compound and the biguanide than by the single administration of the test compound or the biguanide.

### Example 4

Each of the test compound of the aforementioned (1), (3), (56) or (64) and/or an α-glucosidase inhibitor (e.g., voglibose and the like) or a combination of the both pharmaceutical agents was repeatedly administered to ob/ob mice, after which blood glucose level was measured. The results are expressed in the average±standard error of each group. Differences between groups were examined for statistical significance. P<0.05 was considered statistically significant.

The blood glucose level showed greater decrease by the combined administration of the test compound and the α-glucosidase inhibitor than by the single administration of the test compound or the α-glucosidase inhibitor.

### Example 5

Each of the test compound of the aforementioned (1), (3), (56) or (64) and/or an insulin secretagogue (e.g., glibenclamide and the like) or a combination of the both pharmaceutical agents was repeatedly administered to ob/ob mice, after which an oral glucose tolerance test was performed. The results are expressed in the average±standard error of each group. Differences between groups were examined for statistical significance. P<0.05 was considered statistically significant.

Increase in the blood glucose level after glucose tolerance was more strongly suppressed by the combined administration of the test compound and the insulin secretagogue than by the single administration of the test compound or the insulin secretagogue.

### Example 6

Each of the test compound of the aforementioned (1), (3), (56) or (64) and/or a biguanide (e.g., metformin and the like) or a combination of the both pharmaceutical agents was repeatedly administered to ob/ob mice, after which blood glucose level was measured. The results are expressed in the average±standard error of each group. Differences between groups were examined for statistical significance. P<0.05 was considered statistically significant.

The blood glucose level showed greater decrease by the combined administration of the test compound and the biguanide than by the single administration of the test compound or the biguanide.

### Example 7

Placebo and several doses (based on 15 mg/day) of the test compound of the aforementioned (3) were orally administered concurrently with sulfonylurea to type II diabetic patients under treatment with sulfonylureas (glipizide, glyburide or glimepiride) (patients under medication of not less than 50% of the maximum dose recommended in a package insert but failed to sufficiently control diabetes) for plural months.

As a result, HbAlc, which is a parameter of effectiveness, was decreased by not less than 1% point when sulfonylurea and the compound (15 mg/day) of the aforementioned (3) were concurrently administered, and a significant difference was observed in comparison to the single administration of sulfonylurea (concurrent administration of sulfonylurea and placebo).

It was confirmed that, when concurrently administered with sulfonylureas, the test compound proved more effective than the single administration of sulfonylureas.

According to the present invention, diseases such as impaired glucose tolerance disorder, diabetes (e.g., type II diabetes), gestational diabetes, diabetic complications (e.g., diabetic gangrene, diabetic arthropathy, diabetic osteopenia, diabetic glomerulosclerosis, diabetic nephropathy, diabetic dermatopathy, diabetic neuropathy, diabetic cataract, diabetic retinopathy and the like), insulin resistance syndrome (e.g., insulin receptor abnormality, Rabson-Mendenhall syndrome, leprechaunism, Kobberling-Dunnigan syndrome, Lawrence-Seip syndrome (adipose tissue atrophy), Cushing syndrome, acromegaly and the like), polycystic ovary syndrome, hyperlipidemia, atherosclerosis, cardiovascular diseases (e.g., stenocardia, cardiac failure and the like), hyperglycemia (e.g., those characterized by abnormal saccharometabolism such as eating disorders), pancreatitis, osteoporosis, hyperuricemia, hypertension, inflammatory bowel diseases, skin disorders related to an anomaly of differentiation of epidermic cells and the like can be effectively prevented or treated.

This application is based on patent application No. 057555/2004 filed in Japan, the contents of which are hereby incorporated by reference.

While this invention has been shown and described with references to preferred embodiments thereof, it will be understood by those skilled in the art that various changes in form and details may be made therein without departing from the scope of the invention encompasses by the appended claims.

All patents, patent publications and other publications identified or referenced herein are incorporated by reference in their entirety.

1. A pharmaceutical agent comprising, in combination, at least one compound represented by the formula (I):
R¹—SO₂—NH—CO-A¹-CH₂—R² (I)
wherein
R¹ is a group selected from the group consisting of an optionally substituted lower alkyl group, an optionally substituted lower alkenyl group, an optionally substituted aryl group and an optionally substituted heterocyclic group, R² is an optionally substituted aryl group, or an optionally substituted heterocyclic group, and
A¹ is an optionally substituted divalent group of heterobicycle, or a group represented by —CH═CH-A²- wherein A² is an optionally substituted divalent group of nitrogen-containing 5- or 6-membered heterocycle,
or a pharmaceutically acceptable salt thereof, and at least one pharmaceutical agent selected from the group consisting of an α-glucosidase inhibitor, an insulin secretagogue, a sulfonylurea and a biguanide.

2. The pharmaceutical agent of claim 1, wherein R¹ is a lower alkyl group; a lower alkenyl group optionally substituted by an aryl group; an aryl group optionally substituted by a lower alkyl group or a lower alkenyl group; or a heterocyclic group optionally substituted by a halogen atom, and
R² is an aryl group or a heterocyclic group, which group is substituted by at least one group selected from the group consisting of an optionally substituted lower alkyl group, an optionally substituted lower alkenyl group, an optionally substituted lower alkynyl group, an optionally substituted lower alkoxy group, a lower alkylthio group, an optionally substituted amino group, a morpholino group, an acyl group, a halogen atom, an aryl group, an optionally substituted heterocyclic group, and a nitro group.

3. The pharmaceutical agent of claim 1, wherein R¹ is a lower alkyl group, a lower alkenyl group optionally substituted by a phenyl group, a phenyl group optionally substituted by a lower alkyl group or a lower alkenyl group or a halothienyl group,
R² is a group selected from the group consisting of a phenyl group, a naphthyl group and pyridyl group, which group is substituted by at least one group selected from the group consisting of a lower alkyl group optionally substituted by a halogen atom, a phenyl group, a phenoxy group or a cyclo(lower)alkyloxy group; a lower alkenyl group; a lower alkynyl group optionally substituted by a phenyl group; a lower alkoxy group optionally substituted by a phenyl group, a cyclo(lower)alkyl group or a thienyl group; a lower alkylthio group; a lower alkylamino group optionally substituted by a lower alkoxycarbonyl group; a lower alkanoylamino group; a lower alkoxycarbonyl group; a halogen atom; a phenyl group; a thienyl group optionally substituted by a halogen atom; a furyl group; a morpholino group; and a nitro group, and
A¹ is a divalent group of heterobicycle selected from the group consisting of benzimidazole, indole, imidazopyridine, benzofuran and indazole, which group is substituted by at least one lower alkyl group; or A¹ is a group represented by —CH═CH-A²- wherein A² is a divalent group of imidazole, which group is a substituted by at least one group selected from the group consisting of a lower alkyl group and a halogen atom.

4. The pharmaceutical agent of claim 3, wherein the heterobicycle for A¹ is imidazopyridine.

5. The pharmaceutical agent of claim 4, wherein the compound represented by the formula (I) is
3-[(1-bromo-2-naphthyl)methyl]-N-[(5-chloro-2-thienyl)sulfonyl]-2,7-dimethyl-3H-imidazo[4,5-b]pyridine-5-carboxamide, or
ethyl {3-chloro-4-[(2-methyl-5-{[(pentylsulfonyl)amino]carbonyl}-3H-imidazo[4,5-b]pyridin-3-yl)methyl]phenyl}methylcarbamate.

6. The pharmaceutical agent of claim 3, wherein the heterobicycle for A¹ is benzimidazole.

7. The pharmaceutical agent of claim 6, wherein the compound represented by the formula (I) is
1-(2,4-dichlorobenzyl)-2-methyl-N-(pentylsulfonyl)-1H-benzimidazole-6-carboxamide,
1-[(3-chloro-4-biphenylyl)methyl]-2-methyl-N-(pentylsulfonyl)-1H-benzimidazole-6-carboxamide, or
1-(4-bromo-2-chlorobenzyl)-2-methyl-N-[(1E)-1-penten-1-ylsulfonyl]-1H-benzimidazole-6-carboxamide.

8. The pharmaceutical agent of claim 3, wherein the heterobicycle for A¹ is indole.

9. The pharmaceutical agent of claim 8, wherein the compound represented by the formula (I) is
3-[(3-chloro-4-biphenylyl)methyl]-2-methyl-N-(pentylsulfonyl)-1H-indole-5-carboxamide, or
3-[2-chloro-4-(2-thienyl)benzyl]-2-methyl-N-[(4-methylphenyl)sulfonyl]-1H-indole-5-carboxamide.

10. The pharmaceutical agent of claim 3, wherein the heterobicycle for A¹ is benzofuran.

11. The pharmaceutical agent of claim 10, wherein the compound represented by the formula (I) is
3-[(3-chloro-4-biphenylyl)methyl]-2-methyl-N-[(1E)-1-penten-1-ylsulfonyl]-1-benzofuran-5-carboxamide, or
3-[(3-chloro-4-biphenylyl)methyl]-2-methyl-N-{[(E)-2-phenylvinyl]sulfonyl}-1-benzofuran-5-carboxamide.

12. The pharmaceutical agent of claim 3, wherein the heterocycle for A¹ is indazole.

13. The pharmaceutical agent of claim 12, wherein the compound represented by the formula (I) is
1-[(3-chloro-4-biphenylyl)methyl]-3-methyl-N-[(1E)-1-penten-1-ylsulfonyl]-1H-indazole-6-carboxamide.

14. The pharmaceutical agent of claim 3, wherein A¹ is a group represented by —CH═CH-A²- wherein A² is a divalent group of imidazole, which is substituted by at least one group selected from the group consisting of a lower alkyl group and a halogen atom.

15. The pharmaceutical agent of claim 14, wherein the compound represented by the formula (I) is
(2E)-3-{4-chloro-1-[2-chloro-4-(phenylethynyl)benzyl]-2-methyl-1H-imidazol-5-yl}-N-[(4-methylphenyl)sulfonyl]acrylamide,
(2E)-3-{4-chloro-1-[2-chloro-4-(pentyloxy)benzyl]-2-ethyl-1H-imidazol-5-yl}-N-{[(E)-2-phenylvinyl]sulfonyl}acrylamide,
(2E)-3-{4-chloro-1-[2-chloro-4-(phenylethynyl)benzyl]-2-methyl-1H-imidazol-5-yl}-N-(pentylsulfonyl)acrylamide, or
(2E)-3-{4-chloro-1-[2-chloro-4-(phenylethynyl)benzyl]-2-ethyl-1H-imidazol-5-yl}-N-[(1E)-1-penten-1-ylsulfonyl]acrylamide.

16. The pharmaceutical agent of claim 3, wherein the compound represented by the formula (I) is
3-[(1-bromo-2-naphthyl)methyl]-N-[(5-chloro-2-thienyl)sulfonyl]-2,7-dimethyl-3H-imidazo[4,5-b]pyridine-5-carboxamide,
ethyl {3-chloro-4-[(2-methyl-5-{[(pentylsulfonyl)amino]carbonyl}-3H-imidazo[4,5-b]pyridin-3-yl)methyl]phenyl}methylcarbamate,
1-(2,4-dichlorobenzyl)-2-methyl-N-(pentylsulfonyl)-1H-benzimidazole-6-carboxamide,
1-[(3-chloro-4-biphenylyl)methyl]-2-methyl-N-(pentylsulfonyl)-1H-benzimidazole-6-carboxamide,
1-(4-bromo-2-chlorobenzyl)-2-methyl-N-[(1E)-1-penten-1-ylsulfonyl]-1H-benzimidazole-6-carboxamide,
3-[(3-chloro-4-biphenylyl)methyl]-2-methyl-N-(pentylsulfonyl)-1H-indole-5-carboxamide,
3-[(3-chloro-4-biphenylyl)methyl]-2-methyl-N-{[(E)-2-phenylvinyl]sulfonyl}-1-benzofuran-5-carboxamide, or
(2E)-3-{4-chloro-1-[2-chloro-4-(phenylethynyl)benzyl]-2-ethyl-1H-imidazol-5-yl}-N-[(1E)-1-penten-1-ylsulfonyl]acrylamide.

17. The pharmaceutical agent of claim 1, which comprises a compound represented by the formula (I) and an α-glucosidase inhibitor in combination.

18. The pharmaceutical agent of claim 1, which comprises a compound represented by the formula (I) and an insulin secretagogue in combination.

19. The pharmaceutical agent of claim 1, which comprises a compound represented by the formula (I) and a sulfonylurea in combination.

20. The pharmaceutical agent of claim 1, which comprises a compound represented by the formula (I) and a biguanide in combination.

21. A method for preventing and/or treating impaired glucose tolerance disorder, diabetes, gestational diabetes, diabetic complications, insulin resistance syndrome, polycystic ovary syndrome, hyperlipidemia, atherosclerosis, cardiovascular diseases, hyperglycemia, pancreatitis, osteoporosis, hyperuricemia, hypertension, inflammatory bowel diseases, or skin disorders related to an anomaly of differentiation of epidermic cells in a mammal in need thereof, which comprises administering an effective amount of at least one compound represented by the formula (I):
R¹—SO₂—NH—CO-A¹-CH₂—R² (I)
wherein
R¹ is a group selected from the group consisting of an optionally substituted lower alkyl group, an optionally substituted lower alkenyl group, an optionally substituted aryl group and an optionally substituted heterocyclic group,
R² is an optionally substituted aryl group or an optionally substituted heterocyclic group, and
A¹ is an optionally substituted divalent group of heterobicycle or a substituent represented by —CH═CH-A²-, wherein A² is an optionally substituted divalent group of nitrogen-containing 5- or 6-membered heterocycle,
or a pharmaceutically acceptable salt thereof, to said mammal, and
administering, to said mammal, an effective amount of at least one pharmaceutical agent selected from the group consisting of an α-glucosidase inhibitor, an insulin secretagogue, a sulfonylurea and a biguanide.

22. The method of claim 21, which comprises administering, simultaneously, separately or in a staggered manner to said mammal, an effective amount of at least one compound represented by the formula (I) and an effective amount of at least one pharmaceutical agent selected from the group consisting of an α-glucosidase inhibitor, an insulin secretagogue, a sulfonylurea and a biguanide.

23. A commercial package comprising a pharmaceutical agent comprising, in combination, at least one compound represented by the formula (I):
R₁—SO₂—NH—CO-A₁-CH₂—R² (I)
wherein
R¹ is a group selected from the group consisting of an optionally substituted lower alkyl group, an optionally substituted lower alkenyl group, an optionally substituted aryl group and an optionally substituted heterocyclic group,
R² is an optionally substituted aryl group or an optionally substituted heterocyclic group, and
A¹ is an optionally substituted divalent group of heterobicycle or a substituent represented by —CH═CH-A²-, wherein A² is an optionally substituted divalent group of nitrogen-containing 5- or 6-membered heterocycle,
or a pharmaceutically acceptable salt thereof, and
at least one pharmaceutical agent selected from the group consisting of an α-glucosidase inhibitor, an insulin secretagogue, a sulfonylurea and a biguanide, and
a written matter associated with the pharmaceutical agent, the written matter stating that the pharmaceutical agent can or should be used for preventing or treating impaired glucose tolerance disorder, diabetes, gestational diabetes, diabetic complications, insulin resistance syndrome, polycystic ovary syndrome, hyperlipidemia, atherosclerosis, cardiovascular diseases, hyperglycemia, pancreatitis, osteoporosis, hyperuricemia, hypertension, inflammatory bowel diseases or skin disorders related to an anomaly of differentiation of epidermic cells and that an effective amount of at least one compound represented by the formula (I) and an effective amount of at least one pharmaceutical agent selected from the group consisting of an α-glucosidase inhibitor, an insulin secretagogue, a sulfonylurea and a biguanide, can or should be administered, simultaneously, separately or in a staggered manner to a mammal.
